(19) United States
(12) Patent Application Publication
(10) Pub. No.: US 2021/0050092 A1
(43) Pub. Date: Feb. 18, 2021

(54) INTELLIGENT SCHEDULER FOR CENTRALIZED CONTROL OF IMAGING EXAMINATIONS

(71) Applicant: KONINKLIJKE PHILIPS N.V., EINDHOVEN (NL)

(72) Inventors: Carsten Oliver Schirra, Amsterdam (NL); Tanja Nordhoff, Hamburg (DE); Thomas Erik Amthor, Hamburg (DE)

(21) Appl. No.: 16/980,386

(22) PCT Filed: Mar. 14, 2019
(86) PCT No.: PCT/EP2019/056411

§ 371 Date: Sep. 12, 2020

Related U.S. Application Data
(60) Provisional application No. 62/642,694, filed on Mar. 14, 2018.

(51) Int. Cl. G16H 30/20; G16H 40/63

(52) U.S. Cl. CPC G16H 30/20; G16H 40/63

(57) ABSTRACT

Various embodiments of the inventions of the present disclosure a systematic framework of matrices constructed as a basis for a centralized control of assigning imaging operators to operate imaging systems (11) in accordance with a plurality of scheduled imaging examinations. An operator preference matrix (70) including an array of operator preference entries arranged by the imaging operators and the scheduled imaging examinations and an operator availability matrix (80) including an array of operator availability entries arranged by the imaging operators and the scheduled imaging examinations are constructed to provide for a construction of an operator capability matrix (60) including an array of operator capability entries arranged by the imaging operators and the scheduled imaging examinations, which the operator capability matrix (60) serving as a basis for generating an operator assignment schedule (50) for the imaging operators to operate the imaging systems (11) in accordance with the scheduled imaging examinations.

### TECHNICAL FIELD

Various embodiments described in the present disclosure relate to systems, devices and methods for the centralized control of imaging examinations performed by imaging operators locally or remotely operating imaging systems (e.g., X-ray systems, computed tomography systems, magnetic resonance imaging systems, etc.)

### BACKGROUND

Traditionally, an operator of an imaging system executes imaging exams on-site based on a daily prepared exam schedule. As such, to facilitate a productive execution of the scheduled exams by the imaging operator, a queue of a sequential chain of scheduled imaging exams for the operator could be optimized in terms of minimizing idling time of the imaging operator and maximizing any expertise of the imaging operator to promote time efficient, quality imaging exams.

Currently, an imaging operator may locally or remotely concurrently operate an array of imaging systems. As such, a challenge of optimizing a queue of sequential scheduled exams for a single imaging operator changed to a challenge of optimizing a queue of a plurality of scheduled imaging examinations distributed among an ensemble of X imaging operators, X≥2 and Y imaging systems, Y≥2, where the imaging systems may be of the same type/model, the same type/different models and/or of different types.

An idea of centralized control of imaging examinations performed by imaging operators locally or remotely operating imaging systems is premised on evaluating numerous parameters relevant to time efficient, high quality imaging examinations, such as, for example, expertise and availability of each imaging operator, the type of imaging exams to be performed, a profile and availability of patients to be imaged, and the capabilities and operating status of each imaging system. Based on such evaluations, the idea of imaging examinations performed by imaging operators locally or remotely operating imaging systems is further premised on optimizing a queue of the scheduled imaging examinations distributed among the ensemble of X imaging operators and Y imaging systems to promote time efficient, high quality imaging examinations. However, a manual based application of such a centralized control idea is impractical and inferiorly subjective due to limited human cognizance to address a complexity and a dynamic variance of the numerous parameters relevant to time efficient, high quality imaging examinations.

### SUMMARY

Embodiments described in the present disclosure provide a systematic framework of matrices constructed as a basis for a centralized control of assigning imaging operators to operate imaging systems in accordance with a plurality of scheduled imaging examinations. The systematic framework of constructed matrices addresses the complexity and the dynamic variance of the numerous parameters relevant to time efficient, high quality imaging examinations.

Generally, the systematic framework of matrices include (1) an operator preference matrix indicative of a preference for each imaging operator to operate an imaging system for a particular scheduled imaging examination, (2) an operator availability matrix indicative of an availability for each imaging operator to operate an imaging system for a particular scheduled imaging examination, and (3) an operator capability matrix indicative of a capability for each imaging operator to operate an imaging system for a particular scheduled imaging examination, where the capability for each imaging operator is derived from a combination of the operator preference matrix and the operator availability matrix. This systematic framework of matrices facilitates a systematic generation of an operator assignment schedule for the imaging operators to operate the imaging systems in accordance with the scheduled imaging examinations.

One embodiment of the inventions of the present disclosure is an intelligent scheduling controller for optimizing assignments of imaging operators to operate imaging systems in accordance with scheduled imaging examinations. The intelligent imaging scheduling controller comprises a processor and a non-transitory memory configured to (1) construct an operator preference matrix including an array of operator preference entries arranged by a plurality of imaging operators and a plurality of scheduled imaging examinations, wherein each operator preference entry represents a systematic quantification of a preference for a corresponding imaging operator to perform a corresponding scheduled imaging examination, (2) construct an operator availability matrix including an array of operator availability entries arranged by the imaging operators and the scheduled imaging examinations, wherein each operator availability entry represents a systematic quantification of an availability for the corresponding operator to perform the corresponding scheduled imaging examination, (3) construct an operator capability matrix including an array of operator capability entries arranged by the imaging operators and the scheduled imaging examinations, wherein each operator capability entry is a function of a corresponding operator preference entry and a corresponding operator availability entry; and (4) generate an operator assignment schedule for the imaging operators to operate the imaging systems in accordance with the scheduled imaging examinations, wherein the optimized imaging schedule is derived from the operator capability matrix.

A second embodiment of the inventions of the present disclosure is a non-transitory machine-readable storage medium encoded with instructions for execution by a processor for optimizing assignments of a plurality of imaging operators to operate a plurality of imaging systems in accordance with a plurality of scheduled imaging examinations. The non-transitory machine-readable storage medium comprises instructions to (1) construct an operator preference matrix including an array of operator preference entries arranged by a plurality of imaging operators and a plurality of scheduled imaging examinations, wherein each operator preference entry represents a systematic quantification of a preference for a corresponding imaging operator to perform a corresponding scheduled imaging examination, (2) construct an operator availability matrix including an array of operator availability entries arranged by the imaging operators and the scheduled imaging examinations, wherein each operator availability entry represents a systematic quantification of an availability for the corresponding operator to perform the corresponding scheduled imaging examination, (3) construct an operator capability matrix including an array of operator capability entries arranged by the imaging operators and the scheduled imaging examinations, wherein each operator capability entry is a function of a corresponding operator preference entry and a corresponding operator availability entry and (4) generate an operator assignment schedule for the imaging operators to operate the imaging systems in accordance with the scheduled imaging examinations, wherein the optimized imaging schedule is derived from the operator capability matrix.

A third embodiment of inventions of the present disclosure is an intelligent scheduling controller for optimizing assignments of a plurality of imaging operators to operate a plurality of imaging systems in accordance with a plurality of scheduled imaging examinations. The intelligent imaging scheduling method comprising a processor and a non-transitory memory (1) constructing an operator preference matrix including an array of operator preference entries arranged by a plurality of imaging operators and a plurality of scheduled imaging examinations, wherein each operator preference entry represents a systematic quantification of a preference for a corresponding imaging operator to perform a corresponding scheduled imaging examination, (2) constructing an operator availability matrix including an array of operator availability entries arranged by the imaging operators and the scheduled imaging examinations, wherein each operator availability entry represents a systematic quantification of an availability for the corresponding operator to perform the corresponding scheduled imaging examination, (3) constructing an operator capability matrix including an array of operator capability entries arranged by the imaging operators and the scheduled imaging examinations, wherein each operator capability entry is a function of a corresponding operator preference entry and a corresponding operator availability entry and (4) generating an operator assignment schedule for the imaging operators to operate the imaging systems in accordance with the scheduled imaging examinations, wherein the optimized imaging schedule is derived from the operator capability matrix.

For purposes of describing and claiming the inventions of the present disclosure:

(1) the terms of the art of the present disclosure are to be broadly interpreted as known in the art of the present disclosure and exemplary described in the present disclosure;

(2) the term “imaging examination” broadly encompasses any medical procedure involving an imaging of a patient including, but not limited to, imaging scans (e.g., magnetic resonance imaging scans, computed tomography imaging scans, X-ray scans, positron-emission tomography scans and ultrasound scans) and image-guided interventions (e.g., image-guided manual navigation interventions and image-guided robotic navigation interventions).

(2) the term “controller” broadly encompasses all structural configurations, as understood in the art of the present disclosure and as exemplary described in the present disclosure, of an application specific main board or an application specific integrated circuit for controlling an application of various inventive principles of the present disclosure as subsequently described in the present disclosure. The structural configuration of the controller may include, but is not limited to, processor(s), computer-usable/computer readable storage medium(s), an operating system, application module(s), peripheral device controller(s), slot(s) and port(s);

(3) the term “module” broadly encompasses a module incorporated within or accessible by a controller consisting of an electronic circuit and/or an executable program (e.g., executable software stored on non-transitory computer readable medium(s) and/or firmware) for executing a specific application; and

(4) the descriptive labels for term “module” herein facilitates a distinction between modules as described and claimed herein without specifying or implying any additional limitation to the term “module”;

(5) the terms “signal”, “data” and “command” broadly encompasses all forms of a detectable physical quantity or impulse (e.g., voltage, current, magnetic field strength, impedance, color) as understood in the art of the present disclosure and as exemplary described in the present disclosure for transmitting information and/or instructions in support of applying various inventive principles of the present disclosure as subsequently described in the present disclosure. Signal/data/command communication encompassed by the inventions of the present disclosure may involve any communication method as known in the art of the present disclosure including, but not limited to, data transmission/reception over any type of wired or wireless datalink and a reading of data uploaded to a computer-usable/computer readable storage medium; and

(6) the descriptive labels for terms “signal”, “data” and “command” herein facilitates a distinction between signals and data as described and claimed herein without specifying or implying any additional limitation to the terms “signal” and “data”.

The foregoing embodiments and other embodiments of the inventions of the present disclosure as well as various features and advantages of the present disclosure will become further apparent from the following detailed description of various embodiments of the inventions of the present disclosure read in conjunction with the accompanying drawings. The detailed description and drawings are merely illustrative of the inventions of the present disclosure rather than limiting, the scope of the inventions of present disclosure being defined by the appended claims and equivalents thereof.

### BRIEF DESCRIPTION OF THE DRAWINGS

In order to better understand various example embodiments, reference is made to the accompanying drawings, wherein:

FIG. 1 illustrates an exemplary embodiment of an optimized imaging examination system in accordance with the present disclosure;

FIG. 2 illustrates an exemplary embodiment of an optimized magnetic resonance imaging examination system in accordance with the present disclosure;

FIG. 3 illustrates an exemplary embodiment of an intelligent scheduling controller in accordance with the present disclosure;

FIG. 4 illustrates exemplary embodiments of a matrix constructor and a matrix optimizer in accordance with the present disclosure;

FIG. 5 illustrates a flowchart representative of an exemplary embodiment of operator preference matrix generation in accordance with the present disclosure;

FIG. 6 illustrates a flowchart representative of an exemplary embodiment of an operator availability matrix generation in accordance with the present disclosure;

FIG. 7 illustrates a flowchart representative of an exemplary embodiment of an operator capability matrix generation in accordance with the present disclosure;

FIG. 8 illustrates a flowchart representative of an exemplary embodiment of an operator capability matrix optimization in accordance with the present disclosure; and

FIG. 9 illustrates exemplary embodiments of an operator preference matrix, an operator availability matrix and an operator capability matrix in accordance with the present disclosure.

### DETAILED DESCRIPTION

The description and drawings presented herein illustrate various principles. It will be appreciated that those skilled in the art will be able to devise various arrangements that, although not explicitly described or shown herein, embody these principles and are included within the scope of this disclosure. As used herein, the term, “or,” as used herein, refers to a non-exclusive or (i.e., and/or), unless otherwise indicated (e.g., “or else” or “or in the alternative”). Additionally, the various embodiments described in the present disclosure are not necessarily mutually exclusive and may be combined to produce additional embodiments that incorporate the principles described in the present disclosure.

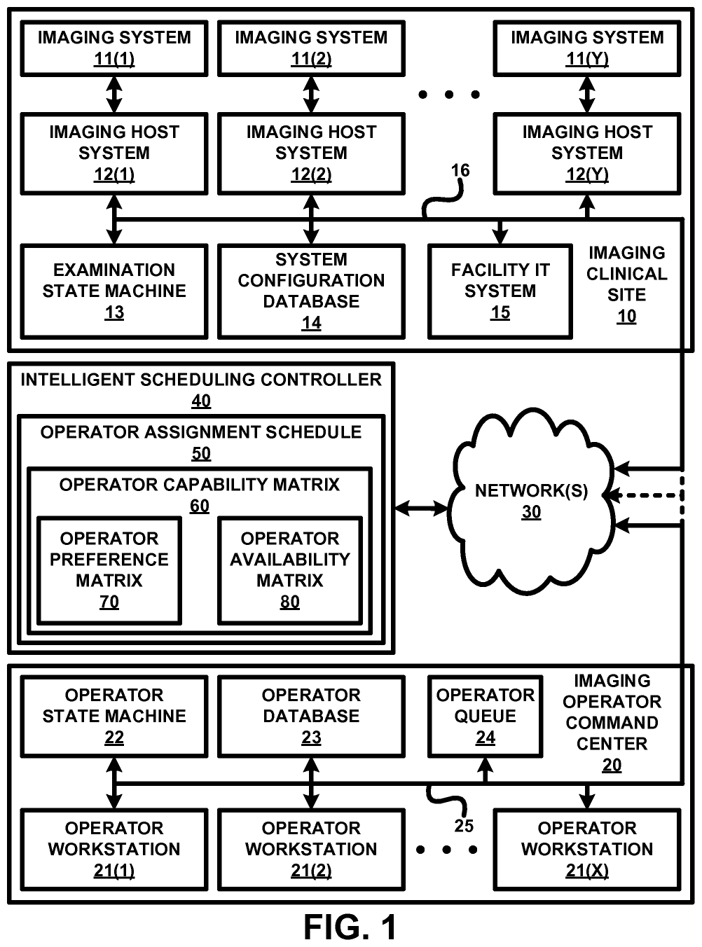

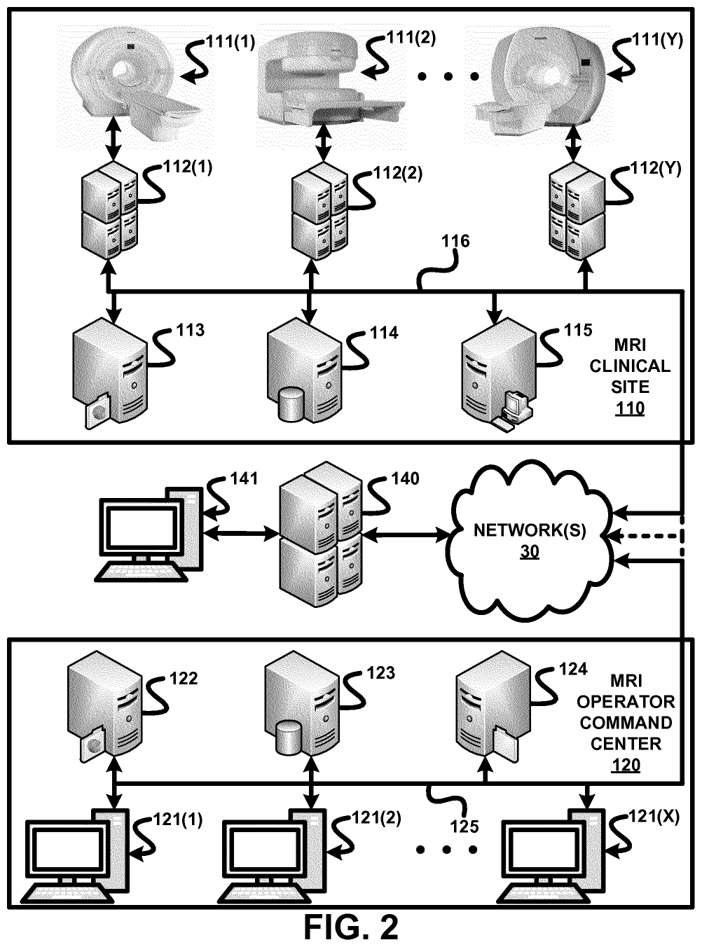

To facilitate an understanding of the inventions of the present disclosure, the following description of FIG. 1 teaches optimized imaging examination system of the present disclosure and FIG. 2 teaches a magnetic resonance imaging system of the present disclosure as an exemplary embodiment of the optimized imaging examination system of FIG. 1. From the description of FIGS. 1 and 2, those having ordinary skill in the art of the present disclosure will appreciate how to apply the present disclosure for making and using numerous and various additional embodiments of optimized imaging examination systems of the present disclosure.

Referring to FIG. 1, an exemplary optimized imaging examination system of the present disclosure encompasses an imaging clinical site 10, an imaging operator command center 20 and an intelligent scheduling controller 40. As shown in FIG. 1, imaging clinical site 10 includes an intranet 16 connected to one or more communication networks 30 (e.g., the Internet, a cellular network, etc.), imaging operator command center 20 includes an intranet 25 connected to network(s) 30, and intelligent scheduling controller 40 is connected to communication network(s) 30.

Alternatively in practice, intranet 16 and intranet 25 may compose a single intranet connected to network(s) 30 as symbolized by the dashed arrow. Furthermore, intelligent scheduling controller 40 may be connected to intranet 16 of imaging clinical site 10 or may be connected to intranet 25 of imaging operator command center 20.

Additionally in practice, imaging clinical site 10, imaging operator command center 20 and intelligent scheduling controller 40 may be physically located at the same location and/or different locations.

Also in practice, additional imaging clinical sites 10 and/or imaging operator command centers 20 may be connected to network(s) 30.

Similarly, referring to FIG. 2, an exemplary optimized magnetic resonance imaging (MRI) examination system of the present disclosure encompasses a MRI clinical site 110, a MRI operator command center 120 and an intelligent scheduling controller 140. As shown in FIG. 2, MRI clinical site 110 includes an intranet 116 connected to one or more communication networks 130 (e.g., the Internet, a cellular network, etc.), MRI operator command center 120 includes an intranet 125 connected to network(s) 130, and intelligent scheduling controller 140 is connected to communication network(s) 130.

Alternatively in practice, intranet 116 and intranet 125 may compose a single intranet connected to network(s) 130 as symbolized by the dashed arrow. Furthermore, intelligent scheduling controller 140 may be connected to intranet 16 of MRI clinical site 110 or may be connected to intranet 125 of MRI operator command center 120.

Additionally in practice, MRI clinical site 110, MRI operator command center 120 and intelligent scheduling controller 140 may be physically located at the same location and/or different locations.

Also in practice, additional MRI clinical sites 110 and/or MRI operator command centers 120 may be connected to network(s) 130.

Referring back to FIG. 1, imaging clinical site 10 employs an Y number of imaging systems 11 as known in of the present disclosure (e.g., X-ray systems, computed tomography systems, magnetic resonance imaging systems, etc.), Y≥2. In one embodiment as shown in FIG. 2, imaging systems 11 are a variety of magnetic resonance imaging scanners 111 located within MRI clinical site 110.

Referring back to FIG. 1, imaging clinical site 10 further employs a Y number of imaging host systems 12, an examination state machine 13, a system configuration database 14 and a facility IT system 15.

Each imaging host system 12 is configured as known in the art of the present disclosure for collecting and logging information and system parameters of an associated imaging system 11, and provides for a current operation state of the associated imaging system 11 at any time. In practice, each host system 12 may be embodied as a software/firmware module that is a component of the associate imaging system 11 or running independently on a server connected to the associated imaging system 11 and intranet 16, such as, for example, on an application server 112 connected to intranet 116 of MRI clinical site 110 as shown in FIG. 2.

Referring back to FIG. 1, examination state machine 13 is a subsystem configured as known in the art of the present disclosure which collects the information of all available imaging host systems 12 to provide information about the ensemble of imaging systems 11 at any time. In practice, examination state machine 13 may be embodied as a software/firmware module as a component of one of the imaging host system 12 or running independently on a server connected to intranet 16, such as, for example, a software/firmware module running on a file transfer protocol server 113 connected to the intranet 116 of MRI clinical site 110 as shown in FIG. 2.

Referring back to FIG. 1, system configuration database 14 is configured as known in the art of the present disclosure to store the current or possible configurations of imaging systems 11. This information includes, but is not limited to, available hardware components of the imaging systems 11, such as, for example, imaging coils of MRI scanners 111 of FIG. 2 for MR imaging. In practice, system configuration database 14 may be embodied as a software/firmware module as a component of examination state machine 13 or running independently on a server connected to intranet 16, such as, for example, a software/firmware module running on a database management server 114 connected to the intranet 116 of MRI clinical site 110 as shown in FIG. 2.

Referring back to FIG. 1, facility IT system 15 provides information about the patients under or scheduled for an imaging examination at imaging clinical site 10 as known in the art of the present disclosure. In practice, facility IT system 15 may be a known IT system (e.g., HIS, RIS or PACS) operating independently on intranet 16, such as, for example, a facility IT system 115 operating as a file management server 115 connected to the intranet 116 of MRI clinical site 110 as shown in FIG. 2.

Referring back to FIG. 1, imaging clinical site 10 as structured results in a data set relevant for intelligent scheduling controller 40 to systematically assign imaging operators to operate imaging systems 11 in accordance with a plurality of scheduled imaging examinations as will be further described in the present disclosure. This clinical data set includes information about the system hardware via examination state machine 13 and system configuration database 14. The clinical data set further includes information about the patients and the exam scheduling via facility IT system 15.

Examples of the system hardware information includes, but is not limited to, (1) an age of each imaging system 11, (2) installed applications on imaging systems 11 (e.g., dedicated heart imaging applications), (3) specialties of each imaging system 11 (e.g., lower patient table for the elderly), (4) imaging systems 11 designated for emergencies and (5) uptimes of each imaging system 11.

Examples of patient information includes, but is not limited to, (1) patient details/availability (e.g., age, gender, implants, physical constraints, BMI, weight, height, anxieties, travel time, time preferences and social detriments of health) and (2) referring details (e.g., medical history and referral source).

Examples of the exam scheduling information includes, but is not limited to, (1) a daily schedule of particular types of scans (e.g., Monday heart scans, Tuesday lung scans, etc.), (2) patterns in imaging times (e.g., Monday afternoons appear to be slower than Tuesday mornings) and (3) time considerations for an age, an anxiety level and a travel time of a patient.

In practice, the clinical data from imaging system 11 and facility IT system 15 may be anonymously transferred to intelligent scheduling controller 40 whereby intelligent scheduling controller 40 may be implemented as an off-site service (e.g., a cloud service) without compromising patient privacy.

Referring back to FIG. 1, imaging operator command center 20 employs an X number of operator workstations 21 configured for operating imaging systems 11 as known in the art of the present disclosure, X≥2. In practice, operator workstations 21 may be embodied in any type of workstation known in the art of the present disclosure, such as, for example, MRI workstations 121 of MRI operator command center 120 consisting of a monitor, a keyboard and a personal computer as shown in FIG. 2.

Referring back to FIG. 1, imaging operator command center 20 further employs an operator state machine 22, an operator database 23 and an operator queue 24.

Operator state machine 22 is a subsystem configured as known in the art of the present disclosure which collects the information of all operators with respect to individual availability to operate one or more imaging systems 11 via an operator workstation 21. In practice, operator state machine 22 may be embodied as a software/firmware module running independently on a server connected to intranet 25, such as, for example, a software/firmware module running on a file transfer protocol server 122 connected to the intranet 125 of MRI operator command center 120 as shown in FIG. 2.

Referring back to FIG. 1, operator database 23 configured as known in the art of the present disclosure to store information about the imaging operators including, but not limited to, performance logging, scan preferences, average scan time per specific exam and operator expertise. In practice, operator database 23 may be embodied as a software/firmware module as a component of operator state machine 22 or running independently on a server connected to intranet 25, such as, for example, a software/firmware module running on a database management server 123 connected to the intranet 125 of MRI operator command center 120 as shown in FIG. 2.

Additionally in practice, operator database 23 may be initialized with an assessment of the imaging operators by supervisor(s) of imaging clinical site 10 and/or with self-assessments by the imaging operators. The operator database thereafter may be automatically updated based on imaging examinations executed by the imaging operators. Such updating includes, but is not limited to, inputting of (1) training meta-data (e.g., total imaging examination duration, number of re-examinations, number of aborted imaging examinations, and selection of protocols versus optimal protocol settings) and (2) evaluation data (e.g., patient satisfaction, imaging operator feedback and staff feedback).

Also in practice, based on the training of each imaging operator, operator database 23 may be further configured to propose specific training to an imaging operator in order to establish and/or increase expertise in certain imaging aspects or associated fields.

Referring back to FIG. 1, operator queue 124 configured as known in the art of the present disclosure to include a current list of scheduled imaging examination per imaging operator and receives updates from intelligent scheduling controller 40. Operator queue 124 may also serve as a front-end system of MRI operator command center 120 to visualize the queueing information and related information (e.g., information about imaging system 11 and key patient data). In practice, operator queue 24 may be may be embodied as a software/firmware module as a component of operator state machine 22 or running independently on a server connected to intranet 25, such as, for example, a software/firmware module running on a file management server 124 connected to the intranet 125 of MRI operator command center 120 as shown in FIG. 2.

Referring back to FIG. 1, imaging operator command center 20 as structured results in a data set relevant for intelligent scheduling controller 40 to systematically assign imaging operators to operate imaging systems 11 in accordance with a plurality of scheduled imaging examinations as will be further described in the present disclosure. This command data set includes information about the operator expertise, preferences, availability and scheduling via operator state machine 22, operator database 23 and operator queue 24.

Examples of operator expertise/preferences (e.g., an operator card) includes, but is not limited to, (1) education background and training level of each imaging operator, (2) year(s) and type(s) of experience of each imaging operator, (3) an image quality assessment of each imaging operator and (4) specialties of each imaging operator (e.g., heart imaging specialist, elderly imaging specialist, contrast agent injection specialist, etc.).

Examples of operator availability and scheduling includes, but is not limited to, (1) weekly schedule of each imaging operator (e.g., imaging operator John Doe normally available only on Mondays and imaging operator Jane Doe normally available on weekends) and (2) full-time and part-time status of each imaging operator.

In practice, the command data from operator state machine 22 and operator database 23 may be anonymously transferred to intelligent scheduling controller 40 in terms of reference numbers of the imaging operators instead of personal information. For this embodiment, intelligent scheduling controller 40 may upload basic information about particular imaging systems 11 and scheduled imaging examinations into operator queue 24 whereby imaging operators may directly communicate personal information to associated imaging host systems 12.

Still referring to FIG. 1, intelligent scheduling controller 40 is configured in accordance with the present disclosure to provide a systematic framework of matrices constructed as a basis for a centralized control of assigning imaging operators to operate imaging systems 11 in accordance with a plurality of scheduled imaging examinations. Generally, the systematic framework of matrices include (1) an operator preference matrix 70 indicative of a preference for each imaging operator to operate an imaging system 11 for a particular scheduled imaging examination, (2) an operator availability matrix 80 indicative of an availability for each imaging operator to operate an imaging system 11 for a particular scheduled imaging examination, and (3) an operator capability matrix 60 indicative of a capability for each imaging operator to operate an imaging system 11 for a particular scheduled imaging examination, where the capability for each imaging operator is derived from a combination of the operator preference matrix 70 and the operator availability matrix 80. This systematic framework of matrices facilitates a systematic generation of an operator assignment schedule 50 for the imaging operators to operate the imaging systems 11 in accordance with the scheduled imaging examinations.

More particularly, to construct operator preference matrix 70, intelligent scheduling controller 40 input information relevant to ascertaining how well each imaging operator may perform on a scheduled imaging examination including, but not limited to, (1) exam scheduling from facility IT systems 15, (2) patient and referral details from facility IT system 15, (3) configuration of imaging systems 11 from system configuration database 14 and (4) operator expertise from operator database 23. As will be further described for exemplary embodiment of intelligent scheduling controller 40 in the present disclosure, the inputted information may be processed by intelligent scheduling controller 40 via a machine learning machine algorithm as known in the art to compute an operator preference score for each imaging operator per each planned imaging examination whereby each operator preference score serves as an entry into operator preference matrix 70. In practice, an operator preference score may be a binary score (e.g., “0” for non-preferred and “1” for preferred) or a level score ranging from a least preference level to a most preferred level (e.g., ranging from “0” least preference level to “1” most preferred level in units of 0.1).

To construct operator availability matrix 80, intelligent scheduling controller 40 inputs information relevant to ascertaining which imaging operators are best situated to perform a particular scheduled imaging examination including, but not limited to, (1) exam scheduling from facility IT systems 15, (2) an availability of each imaging operator from operator state machine 22, (3) an availability of each imaging system 11 from examination state machine 13 and (4) an availability of each patient from facility IT systems 15. As will be further described for exemplary embodiment of intelligent scheduling controller 40 in the present disclosure, the inputted information may be processed by intelligent scheduling controller 40 via a machine learning machine algorithm as known in the art to compute an operator availability score for each imaging operator per each planned imaging examination whereby each operator availability score serves as an entry into operator availability matrix 80. In practice, an operator availability score may be a binary score (e.g., “0” for unavailable and “1” for available) or a level score ranging from a least preference level to a most preferred level (e.g., ranging from “0” least available level to “1” most available level in units of 0.1).

To construct operator capability matrix 60, intelligent scheduling controller 40 combines operator preference matrix 70 and operator availability matrix 80 in a manner to facilitate a generation of operator assignment schedule 50. In practice, a combination of operator preference matrix 70 and operator availability matrix 80 may involve any linear combination technique as known in the art of the present disclosure. As will be further described for exemplary embodiment of intelligent scheduling controller 40 in the present disclosure, a multiplication of matrix elements is preferable for embodiments of operator preference matrix 70 and operator availability matrix 80 having identical tabular arrays whereby an operator capability score is computed from a corresponding operator preference scores and a corresponding operator availability score.

To generate operator assignment schedule 60, intelligent scheduling controller 40 maps a particular imaging operator to each scheduled imaging examination based on operator capability matrix 80. To optimize operator assignment schedule 60, as will be further described for exemplary embodiment of intelligent scheduling controller 40 in the present disclosure, intelligent scheduling controller 40 may execute a multi-dimensional optimization algorithm in mapping a particular imaging operator to each scheduled imaging examination.

In practice, the objective of intelligent scheduling controller 40 may be to construct the matrices 60, 70, 80 and generate the assignment schedule 50 to optimize a particular parameter (e.g., overall imaging time or an imaging quality metric) or a combination of multiple parameters (e.g., combination of overall imaging time and an imaging quality metric).

Additionally in practice, intelligent scheduling controller 40 may be further configured to provide analytics of performance information about imaging systems 11 and the imaging operators. This analytics may be used to as inputs to methods for improving imaging system configurations, imaging operator training, and/or workflow optimization either per imaging system 11 or in the collective.

Also in practice, intelligent scheduling controller 40 may be embodied as a sole controller of a scheduling device or as a component of a scheduling system. For example, as shown in FIG. 2, intelligent scheduling controller 40 may be embodied as a component of a scheduling server 140 that is accessible via a workstation 141 or alternatively, may be a sole controller of workstation 141 or the like (e.g., a laptop or a tablet).

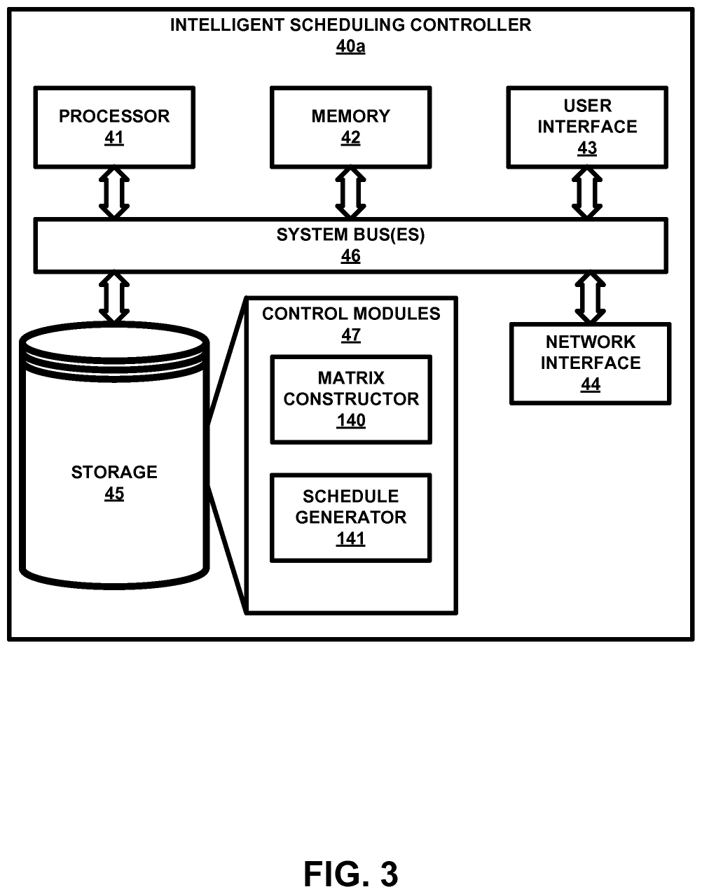

To facilitate a further understanding of the inventions of the present disclosure, the following description of FIGS. 3-9 teaches various embodiments of intelligent scheduling controller 40 of the present disclosure. From the description of FIGS. 3-9, those having ordinary skill in the art of the present disclosure will appreciate how to apply the present disclosure for making and using numerous and various additional embodiments of an intelligent scheduling controller of the present disclosure.

FIG. 3 illustrates an embodiment 40a of intelligent scheduling controller 40 (FIG. 1) to provide a systematic framework of matrices constructed as a basis for a centralized control of assigning imaging operators to operate imaging systems 11 (FIG. 1) in accordance with a plurality of scheduled imaging examinations. As shown, controller 40a includes a processor 41, a memory 42, a user interface 43, a network interface 44, and a storage 45 interconnected via one or more system bus(es) 46. In practice, the actual organization of the components 41-45 of controller 40a may be more complex than illustrated.

The processor 41 may be any hardware device capable of executing instructions stored in memory or storage or otherwise processing data. As such, the processor 41 may include a microprocessor, field programmable gate array (FPGA), application-specific integrated circuit (ASIC), or other similar devices.

The memory 42 may include various memories such as, for example L1, L2, or L3 cache or system memory. As such, the memory 42 may include static random access memory (SRAM), dynamic RAM (DRAM), flash memory, read only memory (ROM), or other similar memory devices.

The user interface 43 may include one or more devices for enabling communication with a user such as an administrator. For example, the user interface 43 may include a display, a mouse, and a keyboard for receiving user commands. In some embodiments, the user interface 43 may include a command line interface or graphical user interface that may be presented to a remote terminal via the network interface 44.

The network interface 44 may include one or more devices for enabling communication with other hardware devices. For example, the network interface 44 may include a network interface card (NIC) configured to communicate according to the Ethernet protocol. Additionally, the network interface 44 may implement a TCP/IP stack for communication according to the TCP/IP protocols. Various alternative or additional hardware or configurations for the network interface will be apparent.

The storage 45 may include one or more machine-readable storage media such as read-only memory (ROM), random-access memory (RAM), magnetic disk storage media, optical storage media, flash-memory devices, or similar storage media. In various embodiments, the storage 45 may store instructions for execution by the processor 41 or data upon with the processor 41 may operate. For example, the storage 45 store a base operating system (not shown) for controlling various basic operations of the hardware.

More particular to the present disclosure, storage 45 further stores control modules 47 including a matrix constructor 140 and a schedule generator 141.

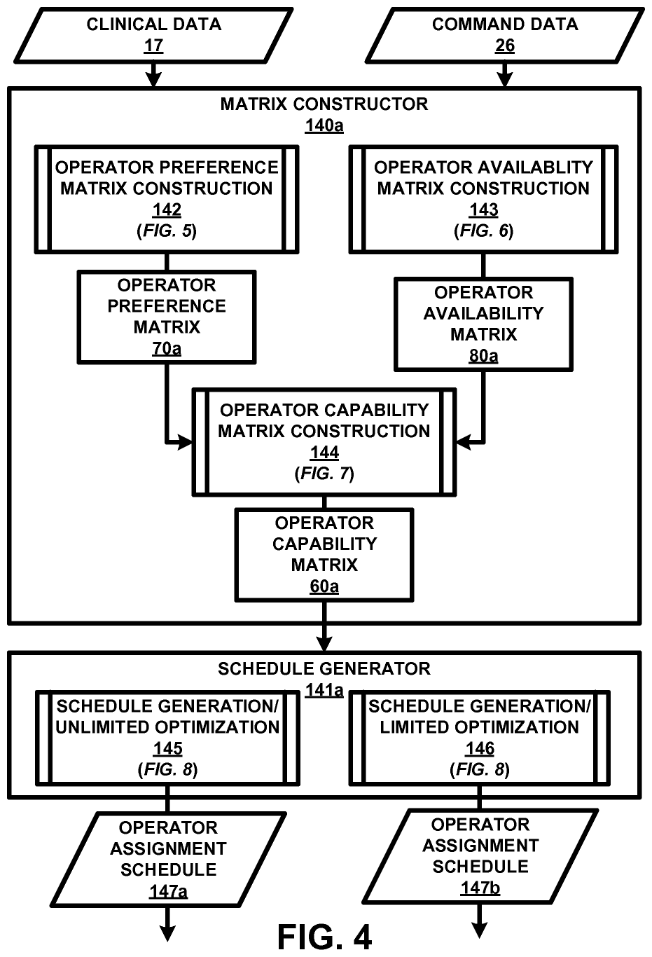

FIG. 4 illustrates an exemplary embodiment 140a of matrix constructor 140 and an exemplary embodiment 141a of schedule generator 141.

Referring to FIG. 4, matrix constructor 140a inputs clinical data 17 from imaging clinical site 10 (FIG. 1) as previously described in the present disclosure and further input command data 26 from imaging command center 20 (FIG. 1) as previously described in the present disclosure. In practice, clinical data 17 and command data 26 may be pushed by clinical site 10 and/or imaging command center 20 to matrix constructor 140a, and/or clinical data 17 and command data 26 may be pulled by matrix constructor 140a from clinical site 10 and/or imaging command center 20.

From the inputted data, matrix constructor 140a sequentially or concurrently executes an operator preference matrix construction 142 for constructing an operator preference matrix 70a and an operator availability matrix construction 143 for constructing an operator availability matrix 80a.

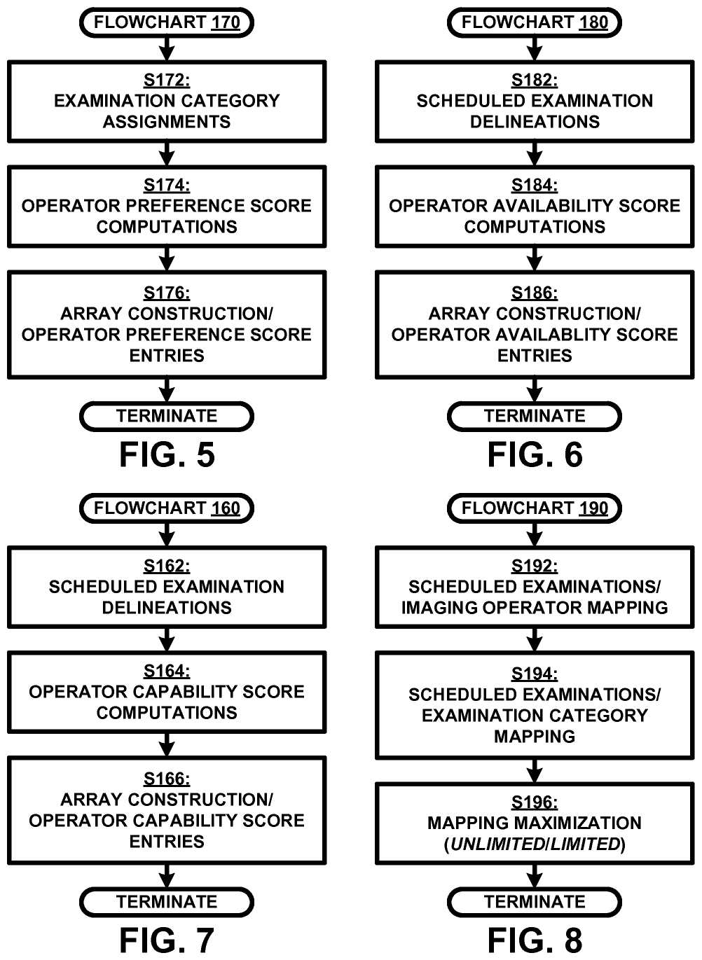

A flowchart 170 as shown in FIG. 5 is an operator preference matrix construction method of the present disclosure implemented by matrix constructor 140a during an execution of operator preference matrix construction 142.

Referring to FIG. 5, a stage S172 of flowchart 170 encompasses matrix constructor 140a assigning each scheduled imaging examinations listed in clinical data 17 to an examination category within columns (or rows) of examination categories. In one embodiment, matrix constructor 140a includes a fixed number of distinct examination categories and assigns each scheduled imaging examination to one of the pre-set examination categories. Examples of such pre-set examination categories include, but are not limited to, anatomical region/structure categories (e.g., liver imaging examinations, brain imaging examinations, cardiac imaging examinations), patient categories (e.g., age, physical constraints, etc.) or a combination of such categories (e.g., liver imaging examinations for patients older than sixty (60) years old and liver imaging examinations for patients sixty (60) years old or younger).

In a second embodiment, encompasses matrix constructor 140a may determine distinct examination categories from the scheduled imaging examinations listed in clinical data 17 and thereafter assign each scheduled imaging examination to an examination category.

In an alternative embodiment, stage S172 of flowchart 170 may encompass a delineation of each schedule imaging examination in columns as (or alternatively in rows) without any assignment to an examination category.

A stage S174 of flowchart 170 encompasses matrix constructor 140a computing operator preference scores for each imaging operator per examination category (or per scheduled imaging examination if examination categories are not utilized). The operator preference score indicates a preference for an imaging operator to conduct imaging examinations under a particular examination category (or to conduct scheduled imaging examinations if examination categories are not utilized). As previously described in the present disclosure, an operator preference score may be a binary score (e.g., “0” for non-preferred and “1” for preferred) or a level score ranging from a least preference level to a most preferred level (e.g., ranging from “0” least preference level to “1” most preferred level in units of 0.1).

Examples of a preference score computation includes, but is not limited to, (1) a preference score of “0” in a liver imaging examination for an imaging operator having zero (0) experience, training and education in liver imaging examination and (2) a preference score of “1” in a liver imaging examination for an imaging operator having over ten (10) years of experience, training and education in liver imaging examinations.

Those of ordinary skill in the art will appreciate the operator preference score computations in practice range from simple assessments to complex evaluations of operator ability to conduct A particular scheduled imaging examination.

A stage S176 of flowchart 170 encompasses a construction of an array of imaging operators (e.g., by operator number or any form of distinctive identification) and scheduled imaging examinations, whereby each operator preference score corresponding to the examination category of each respective scheduled examination serves as an entry into the array.

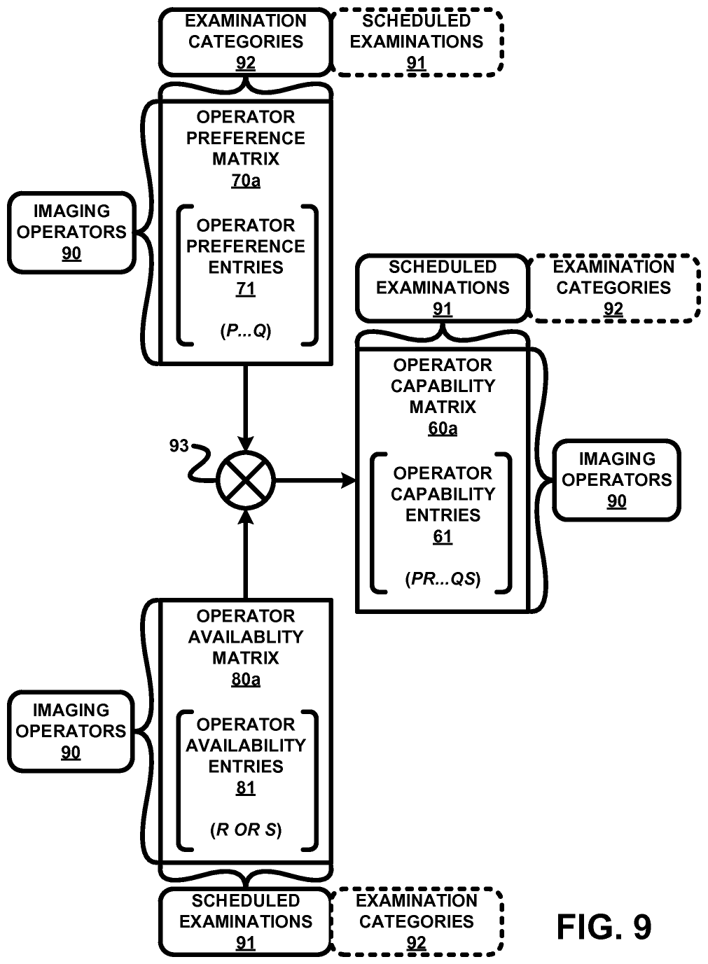

For example, FIG. 9 illustrates a construction of operator preference matrix 70a including an array of imaging operators 90 as rows as shown (or alternatively as columns) and examination categories 92 as columns as shown (or alternatively as rows), whereby each operator preference score serves as an entry into the array. The operator preference entries 71 are scored on a level ranging from a least preference level P to a most preferred level Q (e.g., ranging from “0” least preference level to “1” most preferred level in units of 0.1). Alternatively, a construction of an operator preference matrix may include an array of imaging operators 90 as rows (or alternatively as columns) and scheduled imaging examinations 91 as columns (or alternatively as rows), whereby each operator preference score serves as an entry into the array.

A flowchart 180 as shown in FIG. 6 is an operator availability matrix construction method of the present disclosure implemented by matrix constructor 140a during an execution of operator availability matrix construction 143.

Referring to FIG. 6, a stage S182 of flowchart 180 encompasses matrix constructor 140a delineating each schedule imaging examination in columns as shown (or alternatively in rows).

A stage S184 of flowchart 180 encompasses matrix constructor 140a computing operator availability scores for each imaging operator per scheduled imaging examination. The operator availability score indicates an availability for an imaging operator to conduct particular scheduled imaging examinations. As previously described in the present disclosure, an operator availability score may be a binary score (e.g., “0” for unavailable and “1” for available) or a level score ranging from a least available level to a most available level (e.g., ranging from “0” least available level to “1” most available level in units of 0.1).

Examples of an availability score computation includes, but is not limited to, (1) a availability score of “0” for morning imaging examination for an imaging operator only available in afternoons and (2) a availability score of “1” for morning imaging examination for an imaging operator only available in mornings.

Those of ordinary skill in the art will appreciate the operator availability score computations in practice range from simple assessments to complex evaluations of operator availability to conduct particular scheduled imaging examination.

A stage S186 of flowchart 180 encompasses a construction of an array of imaging operators (e.g., by operator number or any form of distinctive identification) and scheduled imaging examinations, whereby each operator availability score serves as an entry into the array.

For example, FIG. 9 illustrates a construction of operator availability matrix 80a including an array of imaging operators 90 as rows as shown (or alternatively as columns) and scheduled imaging examinations 91 as columns as shown (or alternatively as rows), whereby each operator availability score serves as an entry into the array. The operator availability entries 81 are scored on a level ranging from an unavailable binary level R to an available binary level e.g., “0” for unavailable binary level R and “1” for available binary level S). Alternatively, a construction of an operator availability matrix may include an array of imaging operators 90 as rows (or alternatively as columns) and examination categories 92 as columns (or alternatively as rows), whereby each operator availability score serves as an entry into the array.

Referring back to FIG. 3, upon completion of both operator preference matrix construction 142 and operator availability construction matrix 143, matrix constructor 140a executes an operator capability matrix construction 144.

A flowchart 160 as shown in FIG. 7 is an operator capability matrix construction method of the present disclosure implemented by matrix constructor 140a during an execution of operator capability matrix construction 144 for constructing an operator capability matrix 60a.

Referring to FIG. 7, a stage S162 of flowchart 160 encompasses matrix constructor 140a delineating each schedule imaging examination in columns as shown (or alternatively in rows).

A stage S164 of flowchart 160 encompasses matrix constructor 140a computes an operator capability score of each imaging operator per scheduled imaging examination. As previously described in the present disclosure, a computation of operator capability scores of each imaging operator per scheduled imaging examination may involve any linear combination of operator preference matrix 70a and operator availability matrix 80a as known in the art of the present disclosure.

In one embodiment, an element-wise multiplication of operator preference matrix 70 and operator availability matrix 80 involves an operator capability score being computed from a corresponding operator preference score and a corresponding operator availability score.

A stage S166 of flowchart 160 encompasses a construction of an array of imaging operators (e.g., by operator number or any form of distinctive identification) and scheduled imaging examinations whereby each operator capability score serves as an entry into the array.

For example, FIG. 9 illustrates a construction of operator capability matrix 60a including an array of imaging operators 90 as rows as shown (or alternatively as columns) and scheduled examinations 91 as columns as shown (or alternatively as rows) whereby each operator capability score serves as an entry into the array. For a multiply matrix embodiment, the operator capability entries 61 are scored on a level ranging from least capable PR to most capable QS (e.g., “0” for least capable PR and “1” for most capable QS). Alternatively, a construction of an operator capability matrix may include an array of imaging operators 90 as rows as shown (or alternatively as columns) and examination categories 92 as columns (or alternatively as rows), whereby each operator capability score serves as an entry into the array.

Referring back to FIG. 4, schedule generator 141a inputs and processes operator capability matrix 60a to generate an operator assignment schedule 147a (unlimited optimization) or an operator assignment schedule 147b (limited optimization).

A flowchart 190 as shown in FIG. 8 is an operator assignment schedule generation method of the present disclosure implemented by schedule generator 141a during an execution of a schedule generation 145 (unlimited optimization) 145 or a schedule generation 146 (limited optimization).

Referring to FIG. 8, a stage S192 of flowchart 190 encompasses schedule generator 141a computing an operator map of schedule imaging examinations to each imaging operator. In one embodiment, an operator map m=(2, 1, 2, 3, . . . ) whereby a length of m is the number of scheduled imaging examinations and each entry mᵢ is the number of an imaging operator.

A stage S194 of flowchart 190 encompasses schedule generator 141a mapping scheduled imaging examinations to examination categories (if utilized). In one embodiment, a category map is c=(9, 11, 4, 6, . . . ) whereby a length of c is the number of scheduled imaging examination and each entry cᵢ is the number of an examination category.

A stage S196 of flowchart 190 encompasses schedule generator 141a utilizing a multi-dimensional optimization algorithm as known in the art of the present disclosure (e.g., a Nelder-Mead algorithm, a conjugate gradient algorithm or a Quasi-Newton algorithm) to determine the optimum operator map m that maximizes the sum of capabilities of assigned operators over all scheduled examinations, Σᵢ\hat{M}ₘ_{ᵢ}_{;i}, where \hat{M} is the operator capability matrix.

In one embodiment, the maximization of operator map m does not provide a limitation to the number of simultaneous exams assigned to each imaging operator.

In an alternative embodiment, the optimization of operator map m does provide a limitation to the number of simultaneous exams assigned to each imaging operator. For example, Nⱼ is the maximum number of simultaneous exams assigned to imaging operator j whereby the determination of the maximum of Σᵢ\hat{M}ₘ_{ᵢ}_{;i} is subject to [symbol] Nⱼ<Nₘₐₓ.

Referring back to FIG. 4, operator assignment schedule 147a or operator assignment schedule 147b is uploaded to operator queue 24 (FIG. 1) whereby imaging operators may ascertain their assigned imaging examinations.

Referring to FIGS. 1-9, those having ordinary skill in the art will appreciate the many benefits of the inventions of the present disclosure including, but not limited to, a systematic framework of matrices constructed as a basis for a centralized control of assigning imaging operators to operate imaging systems in accordance with a plurality of scheduled imaging examinations to thereby address the complexity and the dynamic variance of the numerous parameters relevant to time efficient, high quality imaging examinations.

Furthermore, it will be apparent that various information described as stored in the storage may be additionally or alternatively stored in the memory. In this respect, the memory may also be considered to constitute a “storage device” and the storage may be considered a “memory.” Various other arrangements will be apparent. Further, the memory and storage may both be considered to be “non-transitory machine-readable media.” As used herein, the term “non-transitory” will be understood to exclude transitory signals but to include all forms of storage, including both volatile and non-volatile memories.

While the device is shown as including one of each described component, the various components may be duplicated in various embodiments. For example, the processor may include multiple microprocessors that are configured to independently execute the methods described in the present disclosure or are configured to perform steps or subroutines of the methods described in the present disclosure such that the multiple processors cooperate to achieve the functionality described in the present disclosure. Further, where the device is implemented in a cloud computing system, the various hardware components may belong to separate physical systems. For example, the processor may include a first processor in a first server and a second processor in a second server.

It should be apparent from the foregoing description that various example embodiments of the invention may be implemented in hardware or firmware. Furthermore, various exemplary embodiments may be implemented as instructions stored on a machine-readable storage medium, which may be read and executed by at least one processor to perform the operations described in detail herein. A machine-readable storage medium may include any mechanism for storing information in a form readable by a machine, such as a personal or laptop computer, a server, or other computing device. Thus, a machine-readable storage medium may include read-only memory (ROM), random-access memory (RAM), magnetic disk storage media, optical storage media, flash-memory devices, and similar storage media.

It should be appreciated by those skilled in the art that any block diagrams herein represent conceptual views of illustrative circuitry embodying the principles of the invention. Similarly, it will be appreciated that any flow charts, flow diagrams, state transition diagrams, pseudo code, and the like represent various processes which may be substantially represented in machine readable media and so executed by a computer or processor, whether or not such computer or processor is explicitly shown.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

1. An intelligent scheduling controller for optimizing assignments of a plurality of imaging operators to operate a plurality of imaging systems in accordance with a plurality of scheduled imaging examinations, the intelligent scheduling controller comprising a processor and a non-transitory memory configured to:
construct an operator preference matrix including an array of operator preference entries arranged by the imaging operators and the scheduled imaging examinations, wherein each operator preference entry represents a systematic quantification of a preference for a corresponding imaging operator to perform a corresponding scheduled imaging examination;
construct an operator availability matrix including an array of operator availability entries arranged by the imaging operators and the scheduled imaging examinations, wherein each operator availability entry represents a systematic quantification of an availability for the corresponding operator to perform the corresponding scheduled imaging examination;
construct an operator capability matrix including an array of operator capability entries arranged by the imaging operators and the scheduled imaging examinations, wherein each operator capability entry is a function of a corresponding operator preference entry and a corresponding operator availability entry; and
generate an operator assignment schedule for the imaging operators to operate the imaging systems in accordance with the scheduled imaging examinations, wherein the operator assignment schedule is derived from the operator capability matrix.

2. The intelligent scheduling controller of claim 1, wherein a construction of the operator preference matrix includes the processor and the non-transitory memory being configured to:
assign each scheduled imaging examination to one of a plurality of examination categories; and
construct the array of operator preference entries by the imaging operators and the plurality of examination categories representing the scheduled imaging examinations.

3. The intelligent scheduling controller of claim 1, wherein a construction of the operator availability matrix includes the processor and the non-transitory memory being configured to:
assign each scheduled imaging examination to one of a plurality of time slots; and
construct the array of operator availability entries by the imaging operators and the plurality of time slots representing the scheduled imaging examinations.

4. The intelligent scheduling controller of claim 1, wherein a construction of the operator capability matrix includes the processor and the non-transitory memory being configured to:
perform an element-wise multiplication of the operator preference matrix (70) and the operator availability matrix.

5. The intelligent scheduling controller of claim 1, wherein a generation of the operator assignment schedule includes the processor and the non-transitory memory being configured to:
based on the operator capability entries, derive a map of the scheduled imaging examinations to the image operators.

6. The intelligent scheduling controller of claim 5,
wherein the generation of the operator assignment schedule includes the processor and the non-transitory memory being configured to:
apply a limitation to the map of the scheduled imaging examinations to the image operators; and
wherein the limitation represents a maximum number of simultaneous scheduled imaging examination assignable to each imaging operator.

7. The intelligent scheduling controller of claim 1,
wherein a construction of the operator preference matrix includes the processor and the non-transitory memory being configured to;
assign each scheduled imaging examination to one of a plurality of examination categories; and
based on the assignment to examination categories and each operator's preference for the each examination category, derive a map of operator preferences for each combination of operator number and scheduled examination number; and
wherein a generation of the operator assignment schedule includes the processor and the non-transitory memory being configured to:
based on the operator capability entries, deriving at least one of a map of the scheduled imaging examinations to the image operators and a map of the scheduled imaging examinations to the examination categories.

8. The intelligent scheduling controller of claim 7,
wherein the generation of the operator assignment schedule includes the processor and the non-transitory memory being configured to:
apply a limitation to the at least one of the map of the scheduled imaging examinations to the image operators and the map of the scheduled imaging examinations to the examination categories; and
wherein the limitation represents a maximum number of simultaneous scheduled imaging examination assignable to each imaging operator.

9. A non-transitory machine-readable storage medium encoded with instructions for execution by a processor for generating optimized assignments of a plurality of imaging operators to operate a plurality of imaging systems in accordance with a plurality of scheduled imaging examinations, the non-transitory machine-readable storage medium comprising instructions to:
construct an operator preference matrix including an array of operator preference entries arranged by the imaging operators and the scheduled imaging examinations, wherein each operator preference entry represents a systematic quantification of a preference for a corresponding imaging operator to perform a corresponding scheduled imaging examination;
construct an operator availability matrix including an array of operator availability entries arranged by the imaging operators and the scheduled imaging examinations, wherein each operator availability entry represents a systematic quantification of an availability for the corresponding operator to perform the corresponding scheduled imaging examination;
construct an operator capability matrix including an array of operator capability entries arranged by the imaging operators and the scheduled imaging examinations, wherein each operator capability entry is a function of a corresponding operator preference entry and a corresponding operator availability entry; and
generate an operator assignment schedule for the imaging operators to operate the imaging systems in accordance with the scheduled imaging examinations, wherein the operator assignment schedule is derived from an optimization of the operator capability matrix.

10. The non-transitory machine-readable storage medium of claim 9, wherein at least one of:
a construction of the operator preference matrix includes the non-transitory machine-readable storage medium comprising instructions to:
assign each scheduled imaging examination to one of a plurality of examination categories; and
construct the array of operator preference entries by the imaging operators and the plurality of examination categories representing the scheduled imaging examinations; and
a construction of the operator availability matrix includes the non-transitory machine-readable storage medium comprising instructions to:
assign each scheduled imaging examination to one of a plurality of time slots; and
construct the array of operator availability entries by the imaging operators and the plurality of time slots representing the scheduled imaging examinations.

11. The non-transitory machine-readable storage medium of claim 9, wherein a construction of the operator capability matrix includes the non-transitory machine-readable storage medium comprising instructions to:
perform element-wise multiplication of the operator preference matrix and the operator availability matrix.

12. The non-transitory machine-readable storage medium of claim 9, wherein a generation of the operator assignment schedule includes the non-transitory machine-readable storage medium comprising instructions to:
based on the operator capability entries, derive a map of the scheduled imaging examinations to the image operators.

13. The non-transitory machine-readable storage medium of claim 12, wherein the construction of the operator assignment schedule includes the non-transitory machine-readable storage medium comprising instructions to:
apply a limitation to the map of the scheduled imaging examinations to the image operators, wherein the limitation represents a maximum number of simultaneous scheduled imaging examination assignable to each imaging operator.

14. The non-transitory machine-readable storage medium of claim 9,
wherein a construction of the operator preference matrix includes the non-transitory machine-readable storage medium comprising instructions to:
assign each scheduled imaging examination to one of a plurality of examination categories; and
based on the assignment to examination categories and each operator's preference for the each examination category, derive a map of operator preferences for each combination of operator number and scheduled examination number; and
wherein a generation of the operator assignment schedule includes the non-transitory machine-readable storage medium comprising instructions to:
based on the operator capability entries, derive at least one of a map of the scheduled imaging examinations to the image operators and a map of the scheduled imaging examinations to the examination categories.

15. The non-transitory machine-readable storage medium of claim 14, wherein the generation of the operator assignment schedule includes the non-transitory machine-readable storage medium comprising instructions to:
apply a limitation to the at least one of the map of the scheduled imaging examinations to the image operators and the map of the scheduled imaging examinations to the examination categories, wherein the limitation represents a maximum number of simultaneous scheduled imaging examination assignable to each imaging operator.

16. An intelligent scheduling controller for optimizing assignments of a plurality of imaging operators to operate a plurality of imaging systems in accordance with a plurality of scheduled imaging examinations, the intelligent imaging scheduling method comprising a processor and a non-transitory memory:
constructing an operator preference matrix including an array of operator preference entries arranged by the imaging operators and the scheduled imaging examinations, wherein each operator preference entry represents a systematic quantification of a preference for a corresponding imaging operator to perform a corresponding scheduled imaging examination;
constructing an operator availability matrix including an array of operator availability entries arranged by the imaging operators and the scheduled imaging examinations, wherein each operator availability entry represents a systematic quantification of an availability for the corresponding operator to perform the corresponding scheduled imaging examination;
constructing an operator capability matrix including an array of operator capability entries arranged by the imaging operators and the scheduled imaging examinations, wherein each operator capability entry is a function of a corresponding operator preference entry and a corresponding operator availability entry; and
generating an operator assignment schedule for the imaging operators to operate the imaging systems in accordance with the scheduled imaging examinations, wherein the operator assignment schedule is derived from the operator capability matrix.

17. The intelligent imaging scheduling method of claim 16, wherein at least one of:
the constructing of the operator preference matrix includes the processor and the non-transitory memory:
assigning each scheduled imaging examination to one of a plurality of examination categories; and
constructing the array of operator preference entries by the imaging operators and the plurality of examination categories representing the scheduled imaging examinations; and
the constructing of the operator availability matrix includes the processor and the non-transitory memory:
assigning each scheduled imaging examination to one of a plurality of time slots; and
constructing the array of operator availability entries by the imaging operators and the plurality of time slots representing the scheduled imaging examinations.

18. The intelligent imaging scheduling method of claim 16, wherein the constructing of the operator capability matrix includes the processor and the non-transitory memory:
perform element-wise multiplication of the operator preference matrix and the operator availability matrix.

19. The intelligent imaging scheduling method of claim 16, wherein the generating of the operator assignment schedule includes the processor and the non-transitory memory:
based on the operator capability entries, deriving at least one of a map of the scheduled imaging examinations to the image operators and a map of the scheduled imaging examinations to the examination categories; and
applying a limitation to the at least one of the map of the scheduled imaging examinations to the image operators, wherein the limitation represents a maximum number of simultaneous scheduled imaging examination assignable to each imaging operator.

20. The non-transitory machine-readable storage medium of claim 9,
wherein the constructing of the operator preference matrix includes the processor and the non-transitory memory:
assigning each scheduled imaging examination to one of a plurality of examination categories; and
based on the assignment to examination categories and each operator's preference for the each examination category, derive a map of operator preferences for each combination of operator number and scheduled examination number; and
wherein generation of the operator assignment schedule includes the processor and the non-transitory memory:
based on the operator capability entries, deriving a map of the scheduled imaging examinations to the image operators and a map of the scheduled imaging examinations to the examination categories; and
applying a limitation to the map of the scheduled imaging examinations to the image operators and the map of the scheduled imaging examinations to the examination categories, wherein the limitation represents a maximum number of simultaneous scheduled imaging examination assignable to each imaging operator.
